(19) United States
(12) Patent Application Publication
(10) Pub. No.: US 2009/0082638 A1
(43) Pub. Date: Mar. 26, 2009

(54) Measuring Apparatus and Measuring System

(75) Inventor: Yoshihiko Hirao, Nara (JP)

Correspondence Address: RADER FISHMAN & GRAUER PLLC, LION BUILDING, 1233 20TH STREET N.W., SUITE 501, WASHINGTON, DC, 20036, US

(73) Assignee: Yoshihiko Hirano, Kyoto (JP)

(21) Appl. No.: 12/086,113

(22) PCT Filed: Nov. 29, 2006
(86) PCT No.: PCT/JP2006/323758

§ 371 Date: Jun. 5, 2008

(30) Foreign Application Priority Data
Dec. 9, 2005 (JP) 2005-355519

(51) Int. Cl. A61B 5/00

(52) U.S. Cl. 600/300

(57) ABSTRACT

Conventional measuring apparatuses and the like have the problem of not being able to accurately measure information inside the living body. The present invention provides a measuring apparatus disposed inside the living body, comprising: a sensor portion 101 that has an accepting unit 1011 accepting a physical quantity that is to be measured and a signal acquiring unit 1012 acquiring a detection signal, which is a signal corresponding to a result of the acceptance of the accepting unit 1011; a signal output portion 102 that wirelessly outputs the detection signal to the exterior; and a vessel 106 in which at least the signal acquiring unit 1012 of the sensor portion 101 and the signal output portion 102 are sealed.

### TECHNICAL FIELD

The present invention relates to measuring apparatuses and the like arranged inside the living body.

### BACKGROUND ART

As conventional measuring apparatuses, there is an apparatus in which a sensor, a catheter, and the like connected to a measuring apparatus main unit are inserted into the living body, and information obtained via the sensor and the catheter is measured in the measuring apparatus main unit (see Patent Document 1, for example).

### [Patent Document 1] JP 2005-511111A (Tokuhyo) (pp. 15-18, FIGS. 14 to 19, etc.)

### DISCLOSURE OF INVENTION

### Problems to be Solved by the Invention

However, in the conventional measuring apparatuses and the like, information inside the living body is measured using the sensor, the catheter, and the like connected to the measuring apparatus main unit. Thus, during the measurement, for example, the leading end section of the sensor, the catheter, and the like which are connected to the measuring apparatus main unit disposed outside the living body has to be inserted into the living body. Accordingly, for example, during measurement of information inside the human living body, the activities of a person who is subjected to the measurement are restricted (e.g., he or she has to stay in a recumbent position on a bed such that the sensor, the catheter, and the like do not fall off), and thus operations as in daily activities cannot be performed. As a result, there has been the problem that information inside the living body, for example, in a state where daily activities are performed cannot be accurately measured.

Furthermore, since the measurement is performed in a state where a sensor, a catheter, and the like are inserted, the living body is mentally or physically stressed. Thus, the state of the living body is different from the normal state, resulting in the problem that accurate information inside the living body cannot be measured.

A case will be considered as an example in which intravesical pressure is measured in order to diagnose a patient suffering from dysuria or the like. According to a conventional measuring apparatus for measuring pressure in the human bladder, a catheter or the like has to be inserted into the urethra. However, in such a state, the patient cannot walk around, or repeatedly sit down and stand up, as in daily activities. Thus, intravesical pressure information is measured in a resting state, which is different from that in daily activities. The thus obtained result may be different from a change in the intravesical pressure in a state where daily activities are performed. As a result, a change in the intravesical pressure cannot be accurately determined in a state where the patient is performing daily activities, and thus a diagnosis may not be properly given. Furthermore, since the catheter or the like is always inserted into the urethra during the measurement, there are many cases in which measurement of urine flow rate is artificially modified, and the patient is mentally stressed, and feels physical pain and discomfort. Accordingly, the state of the bladder is different from the normal state, and thus it is conceivable that the correlation between the intravesical pressure information and the urinary efficiency cannot be accurately measured. As a result, a diagnosis may not be properly given to the patient.

### Means for Solving the Problems

The present invention is directed to a measuring apparatus disposed inside the living body, comprising: a sensor portion that has an accepting unit accepting a physical quantity that is to be measured and a signal acquiring unit acquiring a detection signal, which is a signal corresponding to the physical quantity accepted by the accepting unit; a signal output portion that wirelessly outputs the detection signal to the exterior; and a vessel in which at least the signal acquiring unit of the sensor portion and the signal output portion are sealed.

With this configuration, the operations of the living body are not restricted. Furthermore, mental or physical stress on the living body due to the measuring apparatus can be reduced. Thus, information inside the living body can be accurately measured. Furthermore, measurement data can be obtained wirelessly in real time. Thus, problems such as errors can be promptly dealt with.

Furthermore, the present invention is directed to a measuring apparatus disposed inside the living body, comprising: a sensor portion that has an accepting unit accepting a physical quantity that is to be measured and a signal acquiring unit acquiring a detection signal, which is a signal corresponding to the physical quantity accepted by the accepting unit; an accumulating portion that accumulates the detection signal; and a vessel in which at least the signal acquiring unit of the sensor portion and the accumulating portion are sealed.

With this configuration, the operations of the living body are not restricted. Furthermore, mental or physical stress on the living body due to the measuring apparatus can be reduced. Thus, information inside the living body can be accurately measured. Furthermore, it is not necessary to provide a structure for wirelessly exchanging information. Thus, the apparatus can be simplified.

Furthermore, in the measuring apparatus of the present invention, a linear member, which is a member in the shape of a line that extends to the exterior, is attached to the vessel.

With this configuration, in a state where the measuring apparatus is disposed inside the living body, if an end of the linear member is disposed outside the living body, it is possible to easily discharge the measuring apparatus out of the living body by pulling this end.

Furthermore, in the measuring apparatus of the present invention, the accepting unit is exposed on the exterior of the vessel.

With this configuration, a physical quantity at a portion that is in the living body or that is in contact with a fluid or mucous membrane inside the living body can be measured.

Furthermore, the measuring apparatus of the present invention further comprises a protector that is shaped so as to cover the accepting unit.

With this configuration, the accepting unit can be prevented from being brought into direct contact with objects, the living body, or the like.

Furthermore, in the measuring apparatus of the present invention, the measuring apparatus is disposed in a fluid inside the living body, and the specific gravity of the measuring apparatus is not greater than that of the fluid.

With this configuration, it is possible to measure a physical quantity of the living body via a fluid, while preventing the measuring apparatus from sinking in the fluid, and to make it difficult for the measuring apparatus to be discharged out of the living body.

Furthermore, in the measuring apparatus of the present invention, the measuring apparatus is disposed in a fluid inside the living body, and the measuring apparatus floats in the fluid.

With this configuration, it is possible to measure a physical quantity of the living body via a fluid, while preventing the measuring apparatus from sinking in the fluid, and to make it difficult for the measuring apparatus to be discharged out of the living body.

Furthermore, in the measuring apparatus of the present invention, the accepting unit is disposed at a position displaced from the center of the vessel, and the center of gravity of the measuring apparatus is positioned closer to the accepting unit.

With this configuration, even if the measuring apparatus swings hard, the upper portion of the measuring apparatus can be always positioned on the upper side of the measuring apparatus.

Furthermore, in the measuring apparatus of the present invention, the accepting unit is disposed in a lower portion in the vessel, and a space is provided in an upper portion in the vessel.

With this configuration, the upper portion of the measuring apparatus is made lighter than the lower portion. Thus, even if the measuring apparatus swings hard, the upper portion of the measuring apparatus can be always positioned on the upper side of the measuring apparatus.

Furthermore, in the measuring apparatus of the present invention, the accepting unit is disposed in the lower portion in the vessel.

With this configuration, the lower portion of the measuring apparatus is made heavier. Thus, even if the measuring apparatus swings hard, the accepting unit can be always positioned in the lower portion in the measuring apparatus. Moreover, if the measuring apparatus is disposed in a fluid or the like, the portion at which the accepting unit of the measuring apparatus is disposed can be kept always in the fluid. Thus, the accepting unit can always accept a physical quantity of the living body via the fluid.

Furthermore, in the measuring apparatus of the present invention, the accepting unit is for accepting pressure.

With this configuration, pressure inside the living body can be measured.

Furthermore, the measuring apparatus of the present invention further comprises: a receiving portion that receives a control signal, which is a signal wirelessly transmitted from the exterior and used for controlling the measuring apparatus; and a control portion that controls the measuring apparatus based on the control signal.

With this configuration, for example, main power of the measuring apparatus can be turned on from the position outside the living body. Thus, the main power of the measuring apparatus can be kept off until the time immediately before the measurement starts, and the power of the power supply portion can be saved.

Furthermore, in the measuring apparatus of the present invention, further comprising: a flexible protection vessel; and a fluid or gel that is sealed together with the vessel in the protection vessel.

With this configuration, a physical quantity of the living body can be measured via the protection vessel and the gel even at a portion where the measuring apparatus cannot be brought into direct contact with the living body.

Furthermore, in the measuring apparatus of the present invention, a linear member that extends to the exterior is attached to the protection vessel.

With this configuration, it is possible to easily discharge the measuring apparatus together with the protection vessel out of the living body, by pulling the linear member.

### EFFECT OF THE INVENTION

With the measuring apparatus and the like according to the present invention, information inside the living body can be accurately measured.

### BEST MODE FOR CARRYING OUT THE INVENTION

Hereinafter, embodiments of a measuring apparatus and the like will be described with reference to the drawings. It should be noted that constituent elements denoted by the same reference numerals in the embodiments perform similar operations, and thus a description thereof may not be repeated.

### Embodiment 1

FIG. 1 is a block diagram of a measuring system in this embodiment. This measuring system includes a measuring apparatus 10 and an information processing apparatus 20. The apparatuses can wirelessly exchange information. The apparatuses are connected to each other, for example, by wireless communications such as Bluetooth (registered trademark) or a wireless LAN. It should be noted that information can be exchanged by means of communications, broadcasting, or the like.

The measuring apparatus 10 includes a sensor portion 101, a signal output portion 102, a power supply portion 103, a receiving portion 104, and a control portion 105. The sensor portion 101 includes an accepting unit 1011 and a signal acquiring unit 1012.

The information processing apparatus 20 includes a signal receiving portion 201, an accumulating portion 202, a control instruction accepting portion 203, and a transmitting portion 204.

The measuring apparatus 10 is disposed inside the living body. The term ‘living body’ refers to the living body of an organism. The term ‘organism’ refers to animals including humans, plants, and the like.

The sensor portion 101 accepts a physical quantity that is to be measured, and acquires a detection signal, which is a signal corresponding to the accepted physical quantity. The term ‘physical quantity that is to be measured’ refers to pressure, acceleration, sound, light, temperature, pH, density of substances, flow rate, current, voltage, and the like, and, in this example, particularly refers to these physical quantities inside the living body. The term ‘physical quantity’ refers to a quantity representing physical properties or states. In this example, a ‘physical quantity’ is, for example, a quantity representing pressure, acceleration, sound, light, temperature, pH, density of substances, flow rate, current, voltage, and the like. More specifically, the sensor portion 101 converts the accepted physical quantity into an electrical signal. There is no limitation on the type of the physical quantity that is to be measured by the sensor portion. Sensors that convert physical quantities of pressure, acceleration, flow rate, and the like into detection signals are usually known respectively as a pressure sensor, an acceleration sensor, a flow sensor, and the like. The basic structure of these sensors is known, and thus a detailed description thereof has been omitted.

In this example, a case will be described as an example in which the sensor portion 101 includes, in particular, the accepting unit 1011 and the signal acquiring unit 1012.

The accepting unit 1011 accepts a physical quantity that is to be measured. The accepting unit 1011 changes, for example, its shape, properties, or the like according to the physical quantity that is to be measured. The accepting unit 1011 functions as a portion that is brought into contact with the living body, a substance inside the living body, or the like, during the measurement, if necessary. The accepting unit 1011 corresponds to, for example, a pressure sensitive diaphragm having piezoresistance or a silicon oscillator, in a pressure sensor or an acceleration sensor.

The signal acquiring unit 1012 acquires a detection signal, which is a signal corresponding to the physical quantity accepted by the accepting unit 1011. More specifically, the signal acquiring unit 1012 converts the physical quantity accepted by the accepting unit 1011, into an electrical signal. For example, the signal acquiring unit 1012 acquires an electrical signal corresponding to a change in the shape or properties of the accepting unit 1011. For example, if the accepting unit 1011 is a pressure sensitive diaphragm having piezoresistance, the signal acquiring unit 1012 takes, as an electrical signal, a change in the resistance of the piezoresistance, which is caused by stress exerted on the piezoresistance based on pressure to the diaphragm. Furthermore, the signal acquiring unit 1012 may include, for example, an amplifying unit such as an amplifier that amplifies a detection signal.

The signal output portion 102 wirelessly outputs a detection signal, more specifically, the detection signal acquired by the sensor portion 101, to the exterior. The term ‘exterior’ used here refers to the exterior of a vessel 106, preferably, the exterior of the living body. Herein, the signal output portion 102 may output the detection signal to the information processing apparatus or the like in the exterior of the vessel 106 disposed inside the living body. There is no limitation on the manner in which the signal output portion 102 outputs the detection signal to the exterior. For example, the detection signal acquired by the signal acquiring unit 1012 may be transmitted as an analog signal, or may be converted into a digital signal and then transmitted. Alternatively, the detection signal may be converted into a sound signal such as tone pulses and then transmitted. Herein, in order to transmit the detection signal as a digital signal, the signal output portion 102 may include an AD converting unit (not shown) that converts an analog detection signal acquired by the sensor portion 101, into a digital signal. The AD converting unit may be included in the sensor portion 101 described above. The signal output portion 102 outputs detection signals at given intervals, more specifically, regular or irregular intervals, for example. The output intervals and the like are determined and set as appropriate based on the precision in accepting physical quantities, the total time necessary for accepting physical quantities, the battery duration, and the like. The signal output portion 102 can be realized as, for example, a combination of driver software for an output device and the output device. For example, the signal output portion 102 is realized by means of wireless communications, more specifically, communications using wireless LAN or the like, short-range wireless communications using Bluetooth or the like, sound output means, etc. Furthermore, the signal output portion 102 may be realized by means of wireless broadcasting. The signal output portion 102 may output the detection signal to a storage medium (not shown) such as a non-volatile memory disposed inside the measuring apparatus 10, and accumulate the detection signal in the storage medium. Herein, the term ‘output’ has a concept that includes outputting a sound, transmission to an external apparatus, accumulation in a storage medium, and the like. Furthermore, the signal output portion 102 may include an antenna (not shown) or the like used for transmitting the detection signal. The signal output portion 102 can be realized as, for example, an integrated circuit such as an LSI (large scale integration).

The power supply portion 103 supplies power to the signal acquiring unit 1012 in the sensor portion 101 and the signal output portion 102. The power may be supplied also to other processing portions and the like in the measuring apparatus 10. Furthermore, the power supply portion 103 may be able to switch on and off the power supplied to the sensor portion 101 and the signal output portion 102, based on instructions and the like from the control portion 105 described later. Furthermore, the power supply portion 103 may be able to switch the status of power supply to other processing portions. The power supply portion 103 may turn the power on and off by accepting instructions other than those from the control portion 105. For example, the power supply portion 103 may be provided with a switch that physically operates, and the power supply portion 103 may supply power if the switch is turned on. If the power supply portion 103 is not provided with a configuration for performing on-and-off control of the power, the sensor portion 101 and the signal output portion 102 may be provided with a switch that can perform control and the like of power supply from the power supply portion 103, based on instructions from the control portion 105. The power supply portion 103 can be realized as, for example, a battery, or a combination of a battery and a switching element. Furthermore, this battery may, or may not, be rechargeable. There is no limitation on the type of the battery, and a lithium battery and the like may be used. It should be noted that the battery preferably is small and can supply power for a long time. Herein, if the power consumption of circuits and the like inside the measuring apparatus 10 is sufficiently small, power may be taken out from radio waves transmitted from the exterior and supplied to the circuits inside the measuring apparatus 10, by configuring the power supply portion 103 so as to perform power supply in a so-called passive RFID tag. Herein, the configuration of the passive RFID tag for taking power out of radio waves is a known art, and thus a description thereof has been omitted.

The receiving portion 104 receives a control signal, which is a signal wirelessly transmitted from the exterior and used for controlling the measuring apparatus 10. Particularly in this example, the control signal transmitted from the information processing apparatus 20 is received. The control signal will be described later. The receiving portion 104 is preferably realized by means of wireless communications, but can be realized also by means of reception of broadcasting. The receiving portion 104 is realized, for example, by means of communications using wireless LAN or the like, short-range wireless communications using Bluetooth or the like (registered trademark), etc. Furthermore, the receiving portion 104 may include an antenna or the like used for receiving the control signal. The receiving portion 104 can be realized as, for example, an integrated circuit such as an LSI. Furthermore, the receiving portion 104 and the signal output portion 102 may be integrated in one integrated circuit or the like. In this case, the antenna or the like may be shared.

The control portion 105 controls the measuring apparatus 10 based on the control signal received by the receiving portion 104. More specifically, the control portion 105 outputs an instruction for controlling the sensor portion 101, the signal output portion 102, the power supply portion 103, and the receiving portion 104 in the measuring apparatus 10, based on the control signal. For example, if the receiving portion 104 receives a control signal for starting the measuring apparatus 10, the control portion 105 outputs, to the power supply portion 103, an instruction for supplying power to the sensor portion 101 and the signal output portion 102. Furthermore, if the receiving portion 104 receives a control signal for calibrating the detection signal that is output by the measuring apparatus 10, the control portion 105 may calibrate the detection signal that is output by the signal acquiring unit 1012, such as zero adjustment, or correction of the detection signal that is output by the signal acquiring unit 1012, based on temperature information or the like contained in the control signal. Furthermore, for example, the receiving portion 104 receives a control signal for resetting the detection signal that is output by the measuring apparatus 10, the control portion 105 may return the detection signal that is output by the signal acquiring unit 1012 to its initial state, such as the factory default state, or may reset the output start time of the signal output portion 102. Furthermore, the remaining power level of the power supply portion 103 may be output based on the control signal received by the receiving portion 104. Herein, the configuration, the method, and the like for calibrating or resetting the detection signal that is output by the sensor and the like are known arts, and thus a detailed description thereof has been omitted. The control portion 105 can be realized typically as an MPU, a memory, or the like. Typically, the processing procedure of the control portion 105 is realized by software, and the software is stored in a storage medium such as a ROM. Note that the processing procedure also may be realized by hardware (dedicated circuit).

Herein, for example, an accumulating portion (not shown) that temporarily accumulates a detection signal and outputs the accumulated detection signal from the signal output portion 102 based on an instruction of the control portion 105 may be provided inside the measuring apparatus 10. Accordingly, the power consumption can be reduced compared with the case in which the detection signal is output at a given timing or the like, and thus the holding electric power in the power supply portion 103 that is included in the measuring apparatus 10 can be reduced. As a result, the power supply portion 103 can be made smaller, and thus the measuring apparatus 10 can be made smaller. The accumulating portion may, or may not, include a storage medium such as a memory or a hard disk in which the detection signal and the like are to be stored. Furthermore, the storage medium may be a non-volatile storage medium, or may be a volatile storage medium. The storage medium may be a storage medium such as a removable flash memory.

The information processing apparatus 20 is disposed typically outside the living body. It should be noted that the information processing apparatus 20 may be partially or entirety embedded inside the living body, if necessary.

The signal receiving portion 201 receives a detection signal. More specifically, the signal receiving portion 201 receives the detection signal wirelessly transmitted from the measuring apparatus 10. The signal receiving portion 201 is preferably realized by means of wireless communications, but can be realized also by means of reception of broadcasting. The signal receiving portion 201 is realized, for example, by means of communications using wireless LAN or the like, short-range wireless communications using Bluetooth or the like, etc. Furthermore, the signal receiving portion 201 may include an antenna or the like used for receiving the detection signal.

The accumulating portion 202 accumulates the detection signal received by the signal receiving portion 201. The accumulating portion 202 stores the received detection signal in a storage medium (not shown) such as a memory or a hard disk. The accumulating portion 202 may, or may not, include this storage medium. The storage medium may be a non-volatile storage medium, or may be a volatile storage medium. It should be noted that the term ‘accumulate’ used here has a concept that also includes temporary storage of data in a storage medium such as a memory, during transmission or reception of a signal, for example.

The control instruction accepting portion 203 accepts a control instruction, which is an instruction for controlling the measuring apparatus 10. The instruction for controlling the measuring apparatus 10 is, for example, an instruction for turning on and off the power of the measuring apparatus 10, an instruction for performing reset, an instruction for performing calibration, an instruction for displaying the remaining battery level, or the like. There is no limitation on the input unit of the control instruction, and a numeric keypad, a keyboard, a mouse, a menu screen, and the like may be used. The control instruction accepting portion 203 can be realized as a device driver of an input unit such as a numeric keypad or a keyboard, control software for a menu screen, or the like.

The transmitting portion 204 transmits a control signal, which is a signal for controlling the measuring apparatus 10 based on the control instruction. For example, a control signal corresponding to a control instruction is stored in advance in a memory or the like, and the transmitting portion 204 acquires the control signal corresponding to the control instruction accepted by the control instruction accepting portion 203, from the memory or the like, and transmits the control instruction to the measuring apparatus 10. The control signal is, for example, a command for the measuring apparatus 10. The transmitting portion 204 is preferably realized by means of wireless communications, but can be realized also by means of broadcasting. The transmitting portion 204 is realized, for example, by means of communications using wireless LAN or the like, short-range wireless communications using Bluetooth or the like, etc. Furthermore, the transmitting portion 204 may include an antenna or the like used for transmitting the control signal.

The processing portion 205 performs a given process on the detection signal accumulated by the accumulating portion 202. The given process may be any process. For example, the given process may be a process of causing a preset analysis program to be executed on the detection signal, a process of judging based on the detection signal whether or not the living body is abnormal, or a process of correcting or calibrating the detection signal. More specifically, the processing portion 205 may output, as a graph, the detection signal accumulated by the accumulating portion 202. For example, it may be judged whether or not a pulse at a given threshold value or more is output in a given section of the detection signal accumulated by the accumulating portion 202, and if such a pulse is output, an analysis result indicating that the living body has an abnormality may be output. The processing portion 205 can be realized typically as an MPU, a memory, or the like. Typically, the processing procedure of the processing portion 205 is realized by software, and the software is stored in a storage medium such as a ROM. Note that the processing procedure also may be realized by hardware (dedicated circuit).

The output portion 206 outputs the detection signal accumulated by the accumulating portion 202 or the detection signal processed by the processing portion 205. The output portion 206 may be considered to include, or to not include, an output device such as a display or a printer. The output portion 206 can be realized as, for example, driver software for an output device, or a combination of driver software for an output device and the output device. Herein, the term ‘output’ has a concept that includes output to a display, output to a printer, transmission to an external apparatus, and the like.

FIG. 2 is a perspective view for illustrating the hardware structure of the measuring apparatus 10 according to this embodiment. FIG. 3 is a cross-sectional view taken along line III-III in the measuring apparatus 10 shown in FIG. 2. In FIGS. 2 and 3, the same reference numerals as those in FIG. 1 denote the same or corresponding portions.

In the vessel 106, at least the signal acquiring unit 1012 of the sensor portion 101 and the signal output portion 102 are sealed. Herein, for example, the signal acquiring unit 1012 of the sensor portion 101, the signal output portion 102, the power supply portion 103, the receiving portion 104, and the control portion 105 are in the interior of the vessel 106, and sealed from the external environment. The internal space of the sealed vessel 106 may be vacuum, or may be filled with a gaseous body such as air or inert gas. If the accepting unit 1011 of the sensor portion 101 has to be brought into contact with the environment or the like inside the living body in order to perform measurement inside the living body, it may be exposed on the exterior of the vessel 106. For example, the accepting unit 1011 may be exposed along the surface of the vessel 106, or the accepting unit 1011 may be projected to the exterior of the vessel 106. In this embodiment, the accepting unit 1011 is exposed on the exterior of the vessel 106, for example, at the lower portion of the vessel 106, particularly in this example, on the bottom face. The circumference of the accepting unit 1011 is sealed to the vessel 106. There is no limitation on the unit that is used for sealing. It should be noted that if the accepting unit 1011 does not have to be brought into contact with the environment or the like inside the living body in order to perform measurement inside the living body, the accepting unit 1011 also may be sealed inside the vessel 106. There is no limitation on the material of the vessel 106, as long as the sealing can be kept, but the material preferably is excellent in corrosion resistance because it is disposed inside the living body. Furthermore, the material of the vessel 106 preferably does not hinder signals from being exchanged. The vessel 106 may be constituted by a single layer or multiple layers made of one material, or may be constituted by multiple layers made of different materials. Furthermore, the material of the vessel 106 preferably is not be damaged by a shock or the like inside the living body. Furthermore, since the vessel 106 is disposed inside the living body, at least the outer portion thereof preferably can resist some sterilization, such as low-temperature sterilization. Since the vessel 106 is disposed inside the living body, the material preferably has biocompatibility. For example, the material used for the outer portion of the vessel 106 preferably is a polymer that has been suitably used for medical tools and the like, such as polyurethane, polystyrene, or ceramics. In this example, the vessel 106 is in the shape of a cylinder as in the case of a capsule or the like in which a drug is to be sealed, as shown in FIGS. 2 and 3, but there is no limitation on the shape. For example, the vessel 106 also may be in the shape of a cone, a column, a torus, or other bodies of rotation whose cross-sectional shape is a circle, a semicircle, an ellipse, a polygon, a polygon with round edges, or the like. Furthermore, the vessel 106 also may be in the shape of a polygonal column. Herein, the edges of the vessel 106 preferably are rounded in order not to damage tissues or the like with which the accepting unit 1011 is brought into contact when it is inserted into the living body. Moreover, the vessel 106 preferably is shaped according to the application. If the measuring apparatus 10 is inserted into the urethra or the like, the vessel 106 preferably has an elongated shape such as an elliptic body of rotation, or other shapes with which the vessel 106 can be easily inserted into a tube or the like. There is no limitation on the size of the vessel 106, but the vessel 106 is preferably small, and, if possible, extremely small in order not to take a toll on the living body in a state where it is inserted into the living body.

If the measuring apparatus 10 is disposed in a fluid inside the living body, such as a bodily fluid or urine, the measuring apparatus 10 preferably has a floating structure in the fluid, that is, a shape, a size, and a weight for floating. The term ‘floating’ used here also refers to a state in which the measuring apparatus 10 is suspended in a fluid without sinking. For example, the specific gravity of the measuring apparatus 10 is preferably equal to or smaller than that of the fluid inside the living body, and more preferably smaller than that of the fluid inside the living body. For example, the vessel 106 is hollow and the space inside the vessel is sufficiently wide, the specific gravity becomes smaller, and thus the measuring apparatus 10 can float in the fluid. The term ‘space’ used here refers to an area in which objects such as processing portions are not arranged except for gaseous bodies. Furthermore, if the weights of the material of the vessel 106, the sensor portion 101, the signal output portion 102, the power supply portion 103 are sufficiently light, the vessel 106 does not have to be hollow, and more specifically, a gap is not necessary between the vessel 106 and processing portions such as the sensor portion 101 or the signal output portion 102. The specific gravity of the measuring apparatus 10 is preferably 0.9 or less, in order to allow the measuring apparatus 10 to float in the fluid inside the living body. Furthermore, the weight of the measuring apparatus 10 preferably is light, in order not to bring a shock or discomfort to the living body in a case where the measuring apparatus 10 hits the living body. For example, the weight is preferably 0.6 g or less.

There is no limitation on the arrangement of the accepting unit 1011, the signal acquiring unit 1012, the signal output portion 102, the power supply portion 103, the receiving portion 104, and the control portion 105, inside the vessel 106. If the accepting unit 1011 is exposed on the exterior of the vessel 106, the arrangement of the accepting unit 1011 is determined according to the position on the vessel 106 where the accepting unit 1011 is to be exposed. Herein, the accepting unit 1011 and the signal acquiring unit 1012 may be integrated on the same substrate, for example, using techniques for manufacturing MEMS (micro electro mechanical systems). For example, one of them may be formed on the surface of the substrate and the other may be formed on the back face of the substrate. Furthermore, the control portion 105, an AD conversion circuit of the signal output portion 102, a switching circuit of the power supply portion 103, or other circuits may be integrated on the same substrate together with the sensor portion 101. Furthermore, the signal output portion 102 and the receiving portion 104 may be integrated on one integrated circuit as a communication module. The manner in which the processing portions are integrated may be changed as appropriate according to the design.

If the measuring apparatus 10 is disposed, in particular, in a fluid inside the living body, it is preferable that the measuring apparatus 10 floats in the fluid as described above, that the accepting unit 1011 is disposed at a position displaced from the center of the measuring apparatus 10, and that the center of gravity of the measuring apparatus 10 is positioned closer to the accepting unit 1011 than the center of the measuring apparatus 10. With this structure, if the measuring apparatus 10 is in a fluid, the side on which the accepting unit 1011 is not disposed faces upward to the fluid surface. Alternatively, the measuring apparatus 10 floats above the fluid surface, and the accepting unit 1011 is always disposed in the fluid. Accordingly, the accepting unit 1011 can always accept a physical quantity inside the living body via the fluid. In order to obtain this configuration, for example, it is only necessary that the sensor portion 101, the signal output portion 102, the power supply portion 103, the receiving portion 104, and the control portion 105 are arranged in the lower portion of the vessel 106, and that a space 30 in which nothing is disposed except for a gaseous body is provided in the upper portion of the vessel 106. In this case, as shown in FIG. 3, the accepting unit 1011 is preferably disposed in the lowest portion of the vessel 106, and exposed on the exterior of the vessel 106 at the lowest portion.

A protector 107 is shaped so as to cover the accepting unit 1011 exposed on the exterior of the vessel 106. The protector 107 is attached to the vessel 106 so as to cover the exposed accepting unit 1011. The protector 107 is provided in order to protect the surface of the accepting unit 1011, to prevent objects from being brought into contact with the accepting unit 1011 and damaging the accepting unit 1011, and to prevent the living body from being brought into contact with the accepting unit 1011 thereby making it impossible to perform accurate measurement. There is no limitation on the shape of the protector 107, as long as the accepting unit 1011 is not hindered from accepting a physical quantity. For example, if the accepting unit 1011 is brought into contact with a fluid inside the living body and accepts a physical quantity relating to the living body from the fluid, the protector 107 may be shaped so as to be capable of preventing the accepting unit 1011 from being brought into contact with a solid or the living body, and to allow the fluid to sufficiently flow onto the surface of the accepting unit 1011. For example, the protector 107 is meshed. In this example, the protector 107 is in the shape of one curved belt. The material of the protector 107 typically is the same as that of the vessel 106, but there is no limitation on the material of the protector 107. Herein, the protector 107 can be omitted, depending on a physical quantity that is to be measured, the arrangement position of the measuring apparatus 10 inside the living body, or the like.

A linear member 108 is a member in the shape of a line extending to the exterior of the vessel 106, and attached to the vessel 106. Herein, the linear member 108 may be attached by bonding or the like to a portion other than the vessel 106 in the measuring apparatus 10, such as the sensor portion 101. The linear member 108 is used for pulling the vessel 106 disposed inside the living body, to the exterior of the living body. Herein, for example, an end of the linear member 108 is tied to the lowest portion of the protector 107 of the vessel 106. There is no limitation on the material and the like of the linear member 108. For example, the linear member 108 may be made of a thread, a rope, a silken gut, a linearly extended polymer, a fine glass fiber, or the like. The linear member 108 preferably is thin, strong, and light, and has a smooth surface. Herein, for example, a nylon thread is used as the linear member 108. The thickness of the linear member 108 is preferably set such that the linear member 108 does not bring discomfort or the like when it is disposed inside the living body or outside the living body. The thickness is preferably 0.5 mm or less. For example, 2-0 (0.3 mm), 1-0 (0.4 mm), or other mononylon threads are preferably used. Furthermore, there is no limitation on the manner in which the linear member 108 is attached to the vessel 106, and bonding and the like may be used. The linear member 108 may be attached to a portion other than the protector 107. The linear member 108 is attached preferably to a portion where the linear member 108 does not hinder the accepting unit 1011 from accepting a physical quantity.

Next, the operation of the measuring apparatus 10 will be described.

If the receiving portion 104 of the measuring apparatus 10 receives a control signal from the exterior, in this example, the information processing apparatus 20, the control portion 105 performs control to turn on the power of the measuring apparatus 10 based on the control signal. Accordingly, power is supplied from the power supply portion 103 to the sensor portion 101 and the signal output portion 102. If power is supplied to the sensor portion 101, the signal acquiring unit 1012 acquires a detection signal by converting the physical quantity relating to the living body, which has been accepted by the accepting unit 1011, into an electrical signal. The signal output portion 102 transmits the detection signal acquired by the signal acquiring unit 1012, to the information processing apparatus 20. The signal output portion 102 may modulate the detection signal, and then transmit it to the information processing apparatus 20. Alternatively, the signal output portion 102 may convert the detection signal into a digital signal, and then transmit it to the information processing apparatus 20. It should be noted that the process of acquiring and transmitting the detection signal is repeatedly performed, until the power is turned off or interruption to end the process takes place.

Next, the operation of the information processing apparatus 20 will be described.

First, it is judged whether or not the signal receiving portion 201 has received a detection signal. If the signal receiving portion 201 has received a detection signal, the accumulating portion 202 accumulates the detection signal received by the signal receiving portion 201, in a storage medium such as a memory. The processing portion 205 performs a given process on the detection signal accumulated by the accumulating portion 202. The output portion 206 outputs the resultant of the process. Then, it is judged again whether or not the signal receiving portion 201 has received a detection signal. Conversely, if the signal receiving portion 201 has not received a detection signal, it is judged whether or not the control instruction accepting portion 203 has accepted a control instruction. If the control instruction accepting portion 203 has not accepted a control instruction, it is judged again whether or not the signal receiving portion 201 has received a detection signal, as described above. If the control instruction accepting portion 203 has accepted a control instruction, the transmitting portion 204 transmits a control signal corresponding to the control instruction, to the measuring apparatus 10. Then, it is judged again whether or not the signal receiving portion 201 has received a detection signal. It should be noted that these processes are repeatedly performed, until the power is turned off or interruption to end the process takes place.

Hereinafter, a specific example of the measuring system in this embodiment will be described. FIG. 4 is a conceptual diagram of the measuring system. Herein, for example, a case will be described in which the measuring apparatus 10 is an apparatus for measuring intravesical pressure in the human bladder.

Herein, for example, the measuring apparatus 10, excluding the protector 107, has an external appearance as a cylinder with round edges as in the case of a drug capsule, as shown in FIGS. 2 and 3. The measuring apparatus 10 is in the shape of a cylinder in this manner for the following reasons. Since the measuring apparatus 10 is disposed in the bladder via the urethra, it is required to make the size of the measuring apparatus 10 as small as possible, in order not to take a toll on the urethra or the like. Since a cylindrical shape allows the height to be freely set to some extent, this shape is preferable in securing an area of the vessel in which the sensor portion 101, the signal output portion 102, the power supply portion 103, and the like can be sufficiently arranged. More specifically, the diameter of the vessel 106 is preferably 4 to 8 mm, and the height thereof is preferably 8 to 17 mm. The space 30 is provided in the upper portion of the vessel 106. The sensor portion 101, the signal output portion 102, the power supply portion 103, the receiving portion 104, and the control portion 105 are arranged closer to the lower portion of the vessel 106. Accordingly, the center of gravity of the measuring apparatus 10 is positioned at the lower portion of the measuring apparatus 10 in which the sensor portion 101 is disposed. The measuring apparatus 10 has a weight and a specific gravity with which the measuring apparatus 10 does not sink in urine. Herein, for example, the shape and the weight of the vessel, and the shapes, the weights, and the like of the sensor portion 101, the signal output portion 102, the power supply portion 103, the receiving portion 104, and the control portion 105 have been adjusted such that the weight of the measuring apparatus 10 is approximately 0.5 g, and the specific gravity thereof is approximately 0.8.

Herein, for example, the sensor portion 101 is a pressure sensor for measuring intravesical pressure. The accepting unit 1011 is exposed on the surface of the measuring apparatus 10 in order to measure pressure.

First, in order to measure intravesical pressure, the measuring apparatus 10 that has been sterilized in advance is inserted into the urethra using a catheter or the like, and the measuring apparatus 10 is disposed inside the bladder. At that time, an end portion of the linear member 108 that is not tied to the measuring apparatus 10 is kept at the exterior through the urethra.

FIG. 5 is a view for illustrating the measuring apparatus 10 disposed inside the bladder. FIG. 5 shows the cross-section of a human body 50 in the vicinity of a bladder 51. As shown in FIG. 5, the measuring apparatus 10 is disposed inside the bladder 51. Since the specific gravity of the measuring apparatus 10 is smaller than that of urine, the measuring apparatus 10 is suspended in a urine 52 in the bladder 51, and stays in the uppermost portion of the bladder 51. Accordingly, it is possible to prevent the measuring apparatus 10 from being discharged together with the urine from the urethra 53 when the urine is discharged. Moreover, the measuring apparatus 10 has the space 30 in the upper portion in the vessel 106, and the center of gravity is positioned at its lower portion in which the sensor portion 101 is disposed. Thus, the measuring apparatus 10 is always suspended in the urine 52 in a state where the sensor portion 101 is at a lower position in the bladder 51. Accordingly, the accepting unit 1011 of the sensor portion 101 exposed on the lower portion of the measuring apparatus 10 is always in contact with the urine 52, and thus the pressure transmitted via the urine 52 can be accepted. In FIG. 5, for the sake of convenience of this description, for example, the scale and the aspect ratio of the measuring apparatus 10 and the human body may be different from the actual values. The same is applicable also to the other drawings.

Next, the user gives a control instruction for turning on the power of the measuring apparatus 10, by operating a menu or the like of the information processing apparatus 20. Based on this control instruction, the transmitting portion 204 of the information processing apparatus 20 outputs, to the measuring apparatus 10, a control signal for instructing to turn the power on.

The receiving portion 104 and the control portion 105 of the measuring apparatus 10 typically operate with very small standby power with which a signal receiving process and the like still can be performed. If a control signal for turning the power on, which has been transmitted from the transmitting portion 204 of the information processing apparatus 20, is received, the control portion 105 controls the power supply portion 103 such that main power is supplied from the power supply portion 103 to the sensor portion 101 and the signal output portion 102.

If the power is supplied, the signal acquiring unit 1012 of the sensor portion 101 acquires a detection signal, which is a signal obtained by converting the physical quantity accepted by the accepting unit 1011 into an electrical signal. Since the accepting unit 1011 is in contact with the urine, if the intravesical pressure increases, the pressure is transmitted via the urine to the accepting unit 1011, and the accepting unit 1011 accepts the information indicating that the pressure has increased. In a similar manner, if the intravesical pressure decreases, the accepting unit 1011 accepts the information indicating that the pressure has decreased. The signal acquiring unit 1012 acquires a detection signal corresponding to the pressure accepted by the accepting unit 1011. Herein, the detection signal may be amplified in an amplifier circuit provided inside the signal acquiring unit 1012.

Even if the measuring apparatus 10 turns sideways and hits the inner wall of the bladder 51 due to a swing motion of the measuring apparatus 10 inside the bladder 51, or even if the amount of urine inside the bladder is reduced due to discharge of the urine, the protector 107 prevents the accepting unit 1011 from being brought into direct contact with the inner wall of the bladder. Accordingly, the accepting unit 1011 is not affected by the pressure generated by contact with the inner wall of the bladder, and thus can be prevented from accepting pressure irrelevant to the intravesical pressure.

The signal output portion 102 samples the detection signal acquired by the signal acquiring unit 1012 at a given timing, converts the detection signal into a digital signal in the AD conversion circuit (not shown) in the signal output portion 102, and wirelessly transmits the converted detection signal to the information processing apparatus 20. The signal output portion 102 may transmit the detection signal to the information processing apparatus 20 each time the detection signal is converted into a digital signal, or may transmit multiple digitized detection signals in a packet. The number of samplings performed by the signal output portion 102 is preferably about 10 times per second in view of the accuracy of a result of the measurement and the power that can be supplied by the power supply portion 103. In the measurement of intravesical pressure, measurement data of the intravesical pressure for approximately 72 hours is typically required in order to give a diagnosis of dysuria or the like. If the measuring apparatus 10 outputs 10 detection signals per second for 72 hours, the power supply portion 103 can be realized, for example, using a cylindrical lithium battery having a diameter of approximately 2 mm and a height of approximately 5 mm.

If the signal receiving portion 201 of the information processing apparatus 20 receives the detection signal from the measuring apparatus 10, the accumulating portion 202 accumulates the received detection signal in a memory or the like. The processing portion 205 reads out the detection signal accumulated by the accumulating portion 202, and performs a given process on the detection signal, herein, for example, a process of constructing data for displaying a graph based on the detection signal. The output portion 206 displays the graph based on the detection signal on a display or the like. FIG. 6 is a display example. In FIG. 6, 1 cmH₂O=98.0665 Pa. On the horizontal axis, the unit on the left of the position indicated by the dotted line represents minute, and the unit on the right of the position represents second. In this example, the number of detection signals received is sequentially counted, and the receiving time at which each detection signal was received is calculated based on the counted number and the information of preset transmission intervals at which the measuring apparatus 10 transmits detection signals. The information processing apparatus 20 may be provided with a clock or the like (not shown), and the accumulating portion 202 may sequentially acquire information of the receiving time at which each detection signal was received based on the clock, and accumulate the information in the memory or the like. Also, the measuring apparatus 10 may be provided with a clock or the like (not shown), the measuring apparatus 10 may transmit a detection signal and information of the detection time at which the detection signal was acquired based on the clock or the like, and the accumulating portion 202 of the information processing apparatus 20 may accumulate the detection signal and the information of the detection time. Herein, the process performed by the processing portion 205 and the output process performed by the output portion 206 may be repeated at regular or irregular given timings according to reception of the detection signals, or may be performed if the signal receiving portion 201 ends reception of the signals or if an instruction is given from the user.

After the measurement performed by the measuring apparatus 10 ends, if the end portion of the linear member 108 kept outside the urethra is pulled, the measuring apparatus 10 follows the linear member 108 from the bladder through the urethra to be discharged to the exterior. In this example, not a wired sensor, a catheter, or the like, but a thin nylon thread or the like is used as the linear member 108. Thus, a person who is having the measuring apparatus 10 inside his or her body feels almost no discomfort due to the linear member 108, and has no problem in performing daily activities.

As described above, according to this embodiment, the measuring apparatus 10 disposed inside the living body detects a physical quantity that is to be measured inside the living body, and wirelessly outputs a detection signal corresponding to a result of the detection, to the exterior of the living body. Thus, a wired sensor or the like does not have to be inserted into the living body in order to detect a physical quantity inside the living body. Accordingly, the operations of the living body are not restricted, and a physical quantity in a state where operations as in daily activities are performed can be detected. Furthermore, mental or physical stress on the living body can be reduced. As a result, accurate information inside the living body can be measured. Furthermore, measurement data can be obtained wirelessly in real time. Thus, problems such as errors occurring can be promptly detected, and dealt with by way of reset or the like.

Furthermore, the measuring apparatus 10 does not sink in a fluid such as urine. Thus, the measuring apparatus 10 stays floating in the interior of an organ or the like, such as the bladder, that is subjected to measurement. Accordingly, even if the fluid is discharged from the lower portion of the organ, the measuring apparatus 10 can be kept inside the organ, by making it difficult for the measuring apparatus 10 to be discharged out of the organ.

Furthermore, the linear member 108 is attached to the measuring apparatus 10. Thus, if the linear member 108 is partially kept outside the living body, the measuring apparatus 10 after the end of detection can be easily discharged out of the living body by pulling the linear member 108.

Herein, in the foregoing specific example, the case was described in which a measuring system is used for measuring intravesical pressure. However, the measuring system according to this embodiment may be used for detecting a physical quantity, not only in the bladder, but also in other organs in which a fluid is kept, such as the stomach, the womb in which a amniotic fluid is kept, and the like. Also in this case, a similar effect to that in the foregoing embodiment can be achieved. More specifically, the measuring apparatus 10 may be disposed inside these organs.

For example, the measuring apparatus 10 described above may be disposed inside the womb. Accordingly, intrauterine pressure can be measured. Herein, in this case, the measuring apparatus 10 does not have to float in a fluid, and thus the specific gravity of the measuring apparatus 10 does not always have to be low enough to allow the measuring apparatus 10 to float in a fluid. If the measuring apparatus 10 is disposed inside the womb in this manner, the accepting unit 1011 in the measuring apparatus 10 may be replaced by an accepting unit that measures sounds, in order to make it possible to measure heartbeats. Alternatively, a sound measurement accepting unit or the like for measuring heartbeats may be provided in the sensor portion 101, in addition to the accepting unit 1011 for measuring pressure. Moreover, a sound measurement sensor portion or the like for measuring heartbeats may be provided in the measuring apparatus 10, in addition to the sensor portion 101 for measuring pressure. The same is applicable also to other embodiments.

Herein, in this embodiment, the measuring apparatus 10 was discharged out of the living body by pulling the linear member 108. For example, if the linear member 108 is not provided, or if the linear member 108 is broken, a foreign-body forceps or the like may be used to remove the measuring apparatus from the living body via the urethra.

Furthermore, in the foregoing specific example, a case was described in which a pressure sensor is used as the sensor portion 101. However, there is no limitation on the sensor used in the present invention. More specifically, a sensor according to a physical quantity that is to be measured may be used.

Furthermore, in this embodiment, the power of the measuring apparatus 10 was turned on and off based on the control signal transmitted from the information processing apparatus 20. However, in the present invention, there is no limitation on the apparatus used for controlling, for example, on and off of the power of the measuring apparatus 10, as long as the apparatus can output a similar control signal to the measuring apparatus 10 as appropriate.

Furthermore, in this embodiment, the case was described in which the measuring apparatus 10 includes the receiving portion 104, the control portion 105, and the like in order to receive the control signal. However, these constituent elements may be omitted if it is not necessary to externally control a power-on state of the measuring apparatus 10. Furthermore, if these constituent elements are omitted, the measuring apparatus 10 may be provided with a switch for turning power on while keeping the sealing state by the vessel 106. For example, if the vessel 106 is flexible, for example, a switch that can be pressed over the vessel 106 may be provided.

Herein, in this embodiment, wireless exchange was used for exchanging information such as detection signals between the measuring apparatus 10 and the information processing apparatus 20. The wavelength and the like of radio waves used in this wireless exchange are preferably set so as not to affect other devices such as a pacemaker.

Furthermore, in the foregoing embodiment, each processing (each function) may be realized by integrated processing using a single apparatus (system), or may be realized by distributed processing using multiple apparatuses. The same is applicable also to other embodiments.

### Embodiment 2

According to this embodiment, a measuring apparatus for measuring intraabdominal pressure is provided in addition to the constituent elements in Embodiment 1.

FIG. 7 is a cross-sectional view for illustrating the hardware structure of the measuring apparatus for measuring intraabdominal pressure in the measuring system according to this embodiment. Hereinafter, the measuring apparatus for measuring intraabdominal pressure is referred to as an ‘intraabdominal pressure measuring apparatus’.

An intraabdominal pressure measuring apparatus 70 includes a measuring portion 11, the linear member 108, a protection vessel 109, a gel 110, and an attachment portion 111. The measuring portion 11 includes the accepting unit 1011, the signal acquiring unit 1012, the signal output portion 102, the power supply portion 103, the receiving portion 104, the control portion 105, and the vessel 106. The measuring portion 11 has a configuration similar to that of the measuring apparatus 10 described with reference to FIGS. 2 and 3 in Embodiment 1, except that the protector and the linear member have been omitted, and a description thereof has been omitted. In this example, the sensor portion 101 of the measuring portion 11 is, for example, a pressure sensor.

The protection vessel 109 is a flexible and hollow vessel that has been sealed. The measuring portion 11 similar to the measuring apparatus 10 shown in FIG. 3 is disposed inside the protection vessel 109. There is no limitation on the shape of the protection vessel 109, as in the case of the vessel 106. There is no limitation on the material of the protection vessel 109, as long as it is a flexible and strong material that is not likely to be broken inside the living body. Available examples of the material of the protection vessel 109 include a polymer film such as a rubber film. Specifically, as the material of the protection vessel 109, a material is used with which the shape of the protection vessel 109 is changed by pressure from the exterior and returns to the original shape when the pressure from the exterior is removed, in a state where the protection vessel 109 is filled with a fluid, a gel, or the like described later. Furthermore, the material preferably has high biocompatibility, and can undergo some sterilization. The size and the shape of the protection vessel 109 are preferably set such that the protection vessel 109 can be inserted through the anus into the rectum. If the protection vessel 109 is in the shape of a sphere, the diameter is preferably approximately 15 to 25 mm.

The protection vessel 109 is filled with the gel 110, and the measuring portion 11 is placed therein. There is no limitation on the gel 110, as long as it is a gel that can efficiently transmit pressure applied to the protection vessel 109, to the measuring portion 11. The gel 110 preferably is a fluid, or a gel having flowability similar to that of a fluid, such that pressure can be uniformly transmitted to the measuring portion 11. The measuring portion 11 is designed so as to have a shape, a specific gravity, and the like with which the measuring portion 11 is suspended in the gel 110 without sinking. As long as pressure applied to the protection vessel 109 can be efficiently transmitted to the measuring portion 11, and the measuring portion 11 does not sink, water or other fluids may be used instead of the gel 110. Furthermore, the fluid may be a viscous fluid, such as a fluid containing polymers.

The attachment portion 111 is a ring-shaped member that is disposed on the protection vessel 109 and to which an end of the linear member 108 is to be tied. The attachment portion 111 may be integrally formed with the protection vessel 109, or may be bonded to the protection vessel 109. In the intraabdominal pressure measuring apparatus 70, the linear member 108 is attached via the attachment portion 111 to the protection vessel 109, in contrast to Embodiment 1. It should be noted that the linear member 108 may be directly attached to the protection vessel 109 by bonding or the like without forming the attachment portion 111.

FIG. 8 is a block diagram showing the configuration of the measuring system according to this embodiment. The configuration of the measuring apparatus 10 and the information processing apparatus 20 is similar to that in the measuring system in Embodiment 1, and thus a detailed description thereof has been omitted. The configuration of the measuring portion 11 of the intraabdominal pressure measuring apparatus 70 is similar to that of the measuring apparatus 10, and thus the same constituent elements are denoted by the same reference numerals and a detailed description thereof has been omitted. It should be noted that since this measuring system includes multiple measuring apparatuses, that is, the measuring apparatus 10 and the intraabdominal pressure measuring apparatus 70, the information processing apparatus 20 is configured so as to be capable of receiving detection signals output by the respective signal output portions 102 and accumulating the detection signals in a classified manner. Alternatively, for example, identifying information is accumulated in association with the detection signals, thereby making it possible to accumulate the detection signals such that they can be classified according to the measuring apparatuses. More specifically, for example, the signal output portions 102 may output identifying information for identifying the measuring apparatuses together with detection signals to the information processing apparatus 20, and in the information processing apparatus 20, for example, the accumulating portion 202 may judge from which measuring apparatus the detection signals have been output, based on the identifying information received together with the detection signals by the signal receiving portion 201, and accumulate the detection signals in a classified manner according to measuring apparatuses. Each of the measuring apparatus 10 and the measuring portion 11 stores in advance the identifying information in a memory or the like. The configuration for accumulating information transmitted from multiple devices in a classified manner is a known art, and thus a detailed description thereof has been omitted.

Herein, the operation of the intraabdominal pressure measuring apparatus 70 is similar to that of the measuring apparatus 10 described in Embodiment 1, and thus a description thereof has been omitted.

Hereinafter, a specific example of the measuring system in this embodiment will be described. The conceptual diagram of this measuring system is the same as that shown in the schematic diagram shown in FIG. 4, except that the intraabdominal pressure measuring apparatus 70 is additionally provided, and thus it is not shown in this example. In this specific example, a case will be described in which the measuring apparatus 10 measures the intravesical pressure inside the human bladder as in Embodiment 1, and, at the same time, the intraabdominal pressure measuring apparatus 70 is used to measure the intraabdominal pressure.

It is known that not only measurement of intravesical pressure, but also deduction of intraabdominal pressure measured at the same time from this intravesical pressure is necessary in order to evaluate accurate activity of the detrusor muscle of the bladder. The reason for this is that a change in the intraabdominal pressure seems to affect also the intravesical pressure. Accordingly, in this embodiment, the intraabdominal pressure is measured at the same time as the measurement of the intravesical pressure. In this example, a value obtained by deducting the intraabdominal pressure from the intravesical pressure is referred to as detrusor muscle contraction pressure.

First, as in Embodiment 1, the measuring apparatus 10 is disposed inside the bladder in order to measure intravesical pressure, and the intraabdominal pressure measuring apparatus 70 is disposed using a finger or the like via the anus into the rectum. At that time, an end of the linear member 108 is kept outside the living body through the anus.

FIG. 9 is a view for illustrating the measuring apparatus 10 disposed inside the bladder and the intraabdominal pressure measuring apparatus 70 disposed inside the rectum. In FIG. 9, the same reference numerals as those in FIG. 5 denote the same or corresponding portions. As shown in FIG. 9, the intraabdominal pressure measuring apparatus 70 is disposed inside a rectum 90. An end of the linear member 108 of the intraabdominal pressure measuring apparatus 70 is extended via an anus 91 to the exterior. The measuring portion 11 inside the intraabdominal pressure measuring apparatus 70 is covered by the protection vessel 109. Thus, the measuring portion 11 is not brought into direct contact with fecal mass, gas, or the like inside the rectum 90, and the measurement of intraabdominal pressure is unlikely to be adversely affected by contact therewith. Moreover, the measuring portion 11 has the space 30 in the upper portion inside the vessel 106, and the center of gravity is positioned at its lower portion in which the sensor portion 101 is disposed. Thus, the measuring portion 11 is always suspended in the gel 110 in a state where the sensor portion 101 is at a lower position in the protection vessel 109. Accordingly, the accepting unit 1011 of the sensor portion 101 exposed on the lower portion of the measuring portion 11 is always in contact with the gel 110, and thus the pressure transmitted via the gel 110 can be accepted.

Next, the user gives a control instruction for turning on the power of the measuring apparatus 10 and the intraabdominal pressure measuring apparatus 70, by operating a menu or the like of the information processing apparatus 20. Based on this control instruction, the transmitting portion 204 of the information processing apparatus 20 outputs, to the measuring apparatus 10 and the intraabdominal pressure measuring apparatus 70, a control signal for instructing to turn the power on.

The receiving portion 104 and the control portion 105 of each of the measuring apparatus 10 and the intraabdominal pressure measuring apparatus 70 typically operate with very small standby power. If a control signal for turning the power on, which has been transmitted from the transmitting portion 204 of the information processing apparatus 20, is received, the control portion 105 controls the power supply portion 103 such that main power is supplied from the power supply portion 103 to the sensor portion 101 and the signal output portion 102.

If the power is supplied, the signal acquiring unit 1012 of the sensor portion 101 acquires a detection signal, which is a signal obtained by converting the physical quantity accepted by the accepting unit 1011 into an electrical signal. The operation in which the measuring apparatus 10 in the bladder acquires a detection signal, and outputs it to the information processing apparatus 20 is similar to that in Embodiment 1, and thus a description thereof has been omitted. Since the accepting unit 1011 of the measuring portion 11 inside the intraabdominal pressure measuring apparatus 70 is in contact with the gel 110, if the intraabdominal pressure increases, the protection vessel 109 is pressed by the rectum 90, this pressure is transmitted via the gel 110 inside the protection vessel 109 to the accepting unit 1011 of the measuring portion 11, and the accepting unit 1011 of the measuring portion 11 accepts the information indicating that the pressure has increased. In a similar manner, if the intraabdominal pressure decreases, the accepting unit 1011 of the measuring portion 11 accepts the information indicating that the pressure has decreased. The signal acquiring unit 1012 of the measuring portion 11 acquires a detection signal corresponding to the pressure accepted by the accepting unit 1011. Herein, the detection signal may be amplified in an amplifier circuit provided inside the signal acquiring unit 1012 of the measuring portion 11.

Since the protection vessel 109 is filled with the gel 110, the measuring portion 11 is unlikely to perform a sudden swing motion. Moreover, even if the measuring portion 11 turns sideways, the gel 110 functions as a buffer, and thus the accepting unit 1011 does not hit hard the inner wall of the protection vessel 109. Accordingly, the accepting unit 1011 is unlikely to be affected by the pressure generated by contact with the inner wall of the protection vessel 109, and thus can be prevented from accepting pressure irrelevant to the intraabdominal pressure.

The signal output portion 102 in the intraabdominal pressure measuring apparatus 70 samples the detection signal acquired by the signal acquiring unit 1012 at a given timing, converts the detection signal into a digital signal in the AD conversion circuit, and wirelessly transmits the converted detection signal to the information processing apparatus 20. The signal output portion 102 may transmit the detection signal to the information processing apparatus 20 each time the detection signal is converted into a digital signal, or may transmit multiple digitized detection signals in a packet.

If the signal receiving portion 201 of the information processing apparatus 20 receives the detection signals from the measuring apparatus 10 and the intraabdominal pressure measuring apparatus 70, the accumulating portion 202 accumulates the received detection signals in the memory or the like with detection signals obtained from the measuring apparatus 10 being classified from detection signals obtained from the intraabdominal pressure measuring apparatus 70. The processing portion 205 reads out the detection signals accumulated by the accumulating portion 202, and performs a given process on the detection signals, herein, for example, a process of constructing data for displaying a graph of intravesical pressure, a graph of intraabdominal pressure, and a graph obtained by deducting intraabdominal pressure from intravesical pressure, that is, a graph of detrusor muscle contraction pressure, based on the detection signals. The output portion 206 displays the graphs based on the detection signals on a display or the like. FIG. 10 is a display example. Herein, the process performed by the processing portion 205 and the output process performed by the output portion 206 may be repeated at regular or irregular given timings according to reception of the detection signals, or may be performed if the signal receiving portion 201 ends reception of the signals or if an instruction is given from the user.

After the measurement of intraabdominal pressure performed by the intraabdominal pressure measuring apparatus 70 ends, if the end portion of the linear member 108 of the intraabdominal pressure measuring apparatus 70 kept outside the living body through the anus is pulled, the intraabdominal pressure measuring apparatus 70 follows the linear member 108 from the rectum through the anus to be discharged to the exterior. In this example, not a wired sensor, a catheter, or the like, but a thin nylon thread or the like is used as the linear member 108. Thus, a person who is having the intraabdominal pressure measuring apparatus 70 inside his or her body feels almost no discomfort due to the linear member 108, and has no problem in performing daily activities.

As described above, according to this embodiment, a similar effect to that in Embodiment 1 is achieved because the measuring apparatus 10 is used. Furthermore, the intraabdominal pressure measuring apparatus 70 disposed inside the living body detects a physical quantity that is to be measured inside the living body, and wirelessly outputs a detection signal corresponding to a result of the detection, to the exterior of the living body. Thus, a wired sensor or the like does not have to be inserted into the living body in order to detect a physical quantity inside the living body. For example, in conventional measurement of intraabdominal pressure, a balloon is disposed inside the rectum, and a wired sensor disposed inside the balloon is used to measure intraabdominal pressure transmitted inside the balloon. Thus, as in the case where intravesical pressure is measured, operations are restricted, and the living body is mentally or physically stressed. Accordingly, accurate measurement cannot be performed. However, according to this embodiment, with the configuration described above, the operations of the living body are not restricted, and a physical quantity in a state where operations as in daily activities are performed can be detected. Furthermore, mental or physical stress on the living body can be reduced. As a result, accurate information inside the living body can be measured.

Furthermore, intravesical pressure and intraabdominal pressure can be measured at the same time, and thus accurate activities and the like of the detrusor muscle of the bladder can be evaluated.

Furthermore, intraabdominal pressure is transmitted via the protection vessel 109 and the gel 110 to the accepting unit 1011. Thus, the accepting unit 1011 is not brought into direct contact with fecal mass or gas inside the rectum. Accordingly, it is possible to prevent the measurement problems that erroneous detection is performed due to contact with these materials, or that detection becomes impossible due to fecal mass covering the accepting unit 1011, for example.

Furthermore, the linear member 108 is attached to the intraabdominal pressure measuring apparatus 70. Thus, if the linear member 108 is partially kept outside the living body, the intraabdominal pressure measuring apparatus 70 after the end of detection can be easily discharged out of the living body by pulling the linear member 108.

Herein, in the foregoing embodiments, the case was described as an example in which the signal output portion 102 transmits a detection signal to the information processing apparatus 20 disposed outside the living body. However, in the present invention, as in the case of a measuring apparatus 10a shown in FIG. 11, a non-volatile storage medium 1100 such as a flash ROM in which information can be stored may be provided in the measuring apparatus 10a, and the signal output portion 102 may accumulate a detection signal in the storage medium 1100 instead of transmitting the detection signal to the exterior. In this case, after the measurement ends, a detection signal as a result of the measurement can be acquired by discharging the measuring apparatus 10a out of the living body, opening the vessel 106, taking out the storage medium 1100, and reading out the detection signal accumulated in the storage medium 1100 using a reading apparatus or the like for the storage medium 1100. In this case, for example, the measuring apparatus 10a may be provided with a clock portion (not shown) such as a clock for measuring time, and the signal output portion 102 may accumulate the detection signal together with information of the time at which the detection signal was obtained based on the clock portion, in the storage medium 1100. With this configuration, it is not necessary to provide, for example, a communication unit for transmitting the detection signal of the measuring apparatus, or an apparatus for receiving the detection signal. It will be appreciated that this configuration is applicable also to the measuring portion 11.

Furthermore, in this embodiment, the case was described in which the intraabdominal pressure measuring apparatus 70 is used for measuring intraabdominal pressure. However, a measuring apparatus having a configuration similar to that of the intraabdominal pressure measuring apparatus 70 may be used for measurements other than that of intraabdominal pressure. For example, with such a measuring apparatus, a physical quantity of the living body can be measured even at a portion where the measuring portion cannot be brought into direct contact with the living body. Thus, a similar effect to that in the foregoing embodiment can be achieved.

For example, the apparatus having a configuration similar to that of the intraabdominal pressure measuring apparatus 70 described above may be disposed inside the womb. Accordingly, intrauterine pressure can be measured. If the apparatus is deposed inside the womb in this manner, the accepting unit 1011 in the measuring portion 11 may be replaced by an accepting unit that measures sounds, in order to make it possible to measure heartbeats. Alternatively, a sound measurement accepting unit or the like for measuring heartbeats may be provided in the sensor portion 101, in addition to the accepting unit 1011 for measuring pressure. Moreover, a sound measurement sensor portion or the like for measuring heartbeats may be provided in the measuring portion 11, in addition to the sensor portion 101 for measuring pressure. Alternatively, a measuring apparatus in which the accepting unit 1011 in the measuring portion 11 is replaced by an accepting unit that measures sounds may be provided in the intraabdominal pressure measuring apparatus 70, in addition to the measuring portion 11.

Furthermore, in this embodiment, it will be appreciated that the intraabdominal pressure measuring apparatus 70 may be used alone separately from the measuring apparatus 10.

Furthermore, in the foregoing embodiments, it will be appreciated that two or more communication units (the signal output portion, the receiving portion, etc.) in one apparatus may be physically realized as one medium. The same is applicable also to other embodiments.

The present invention is not limited to the embodiments set forth herein. Various modifications are possible within the scope of the present invention.

### INDUSTRIAL APPLICABILITY

As described above, the measuring apparatus and the like according to the present invention are suitable as an apparatus and the like for measuring a physical quantity inside the living body, and in particular, they are useful as an measuring apparatus and the like arranged in a fluid inside the living body.

### BRIEF DESCRIPTION OF THE DRAWINGS

FIG. 1 is a block diagram of a measuring system in Embodiment 1.

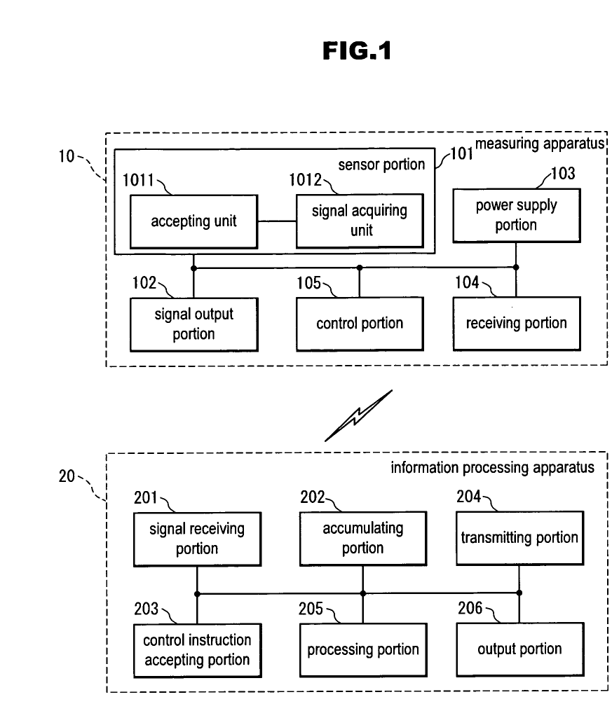

FIG. 2 is a perspective view of a measuring apparatus in the measuring system.

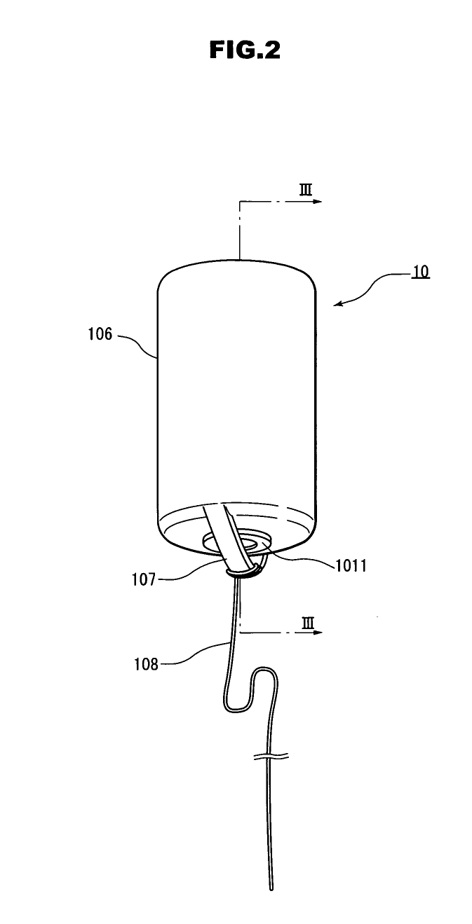

FIG. 3 is a cross-sectional view of the measuring apparatus.

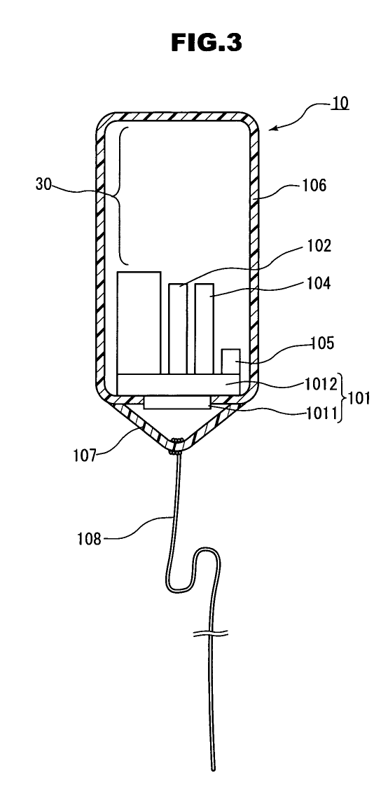

FIG. 4 is a conceptual diagram of the measuring system.

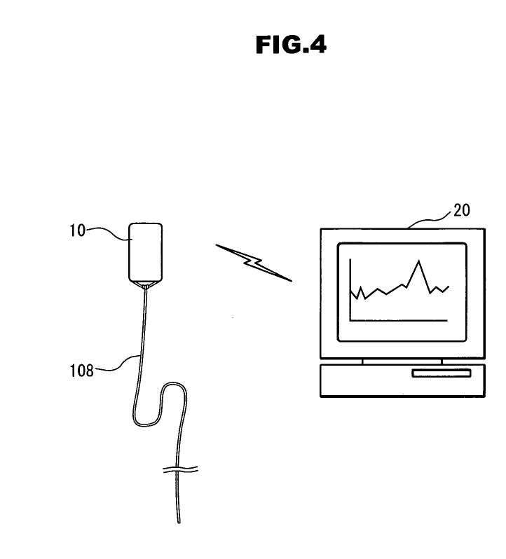

FIG. 5 is a view showing an arrangement example of the measuring apparatus inside the living body.

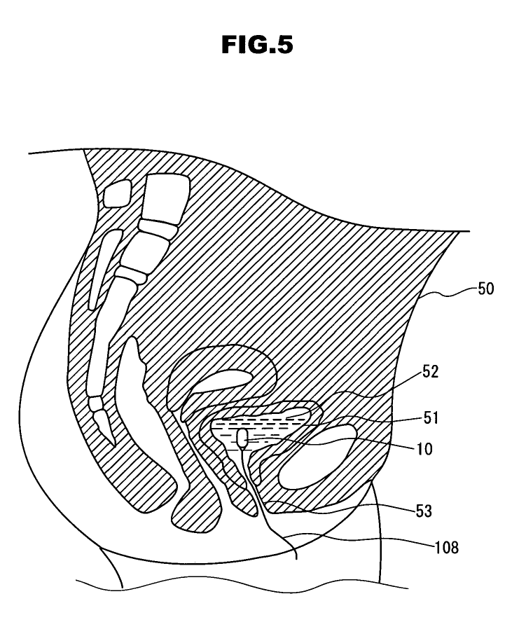

FIG. 6 is a graph showing a display example in the measuring system.

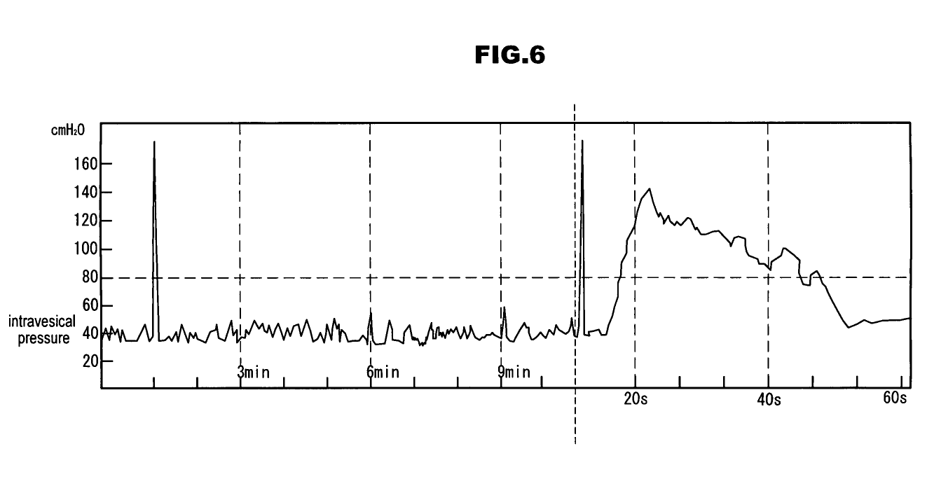

FIG. 7 is a cross-sectional view of an intraabdominal pressure measuring apparatus in a measuring system in Embodiment 2.

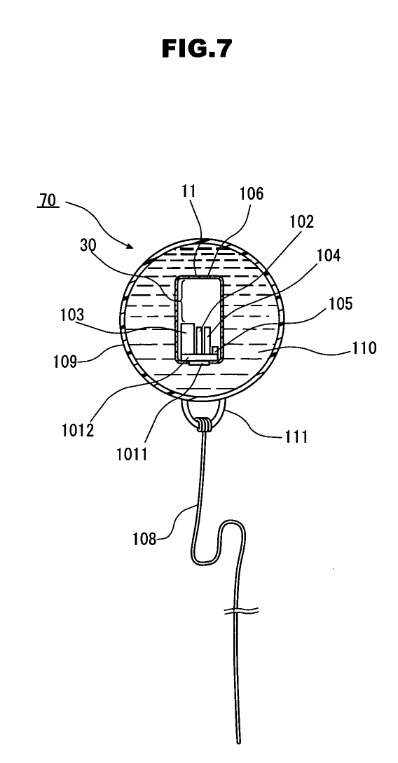

FIG. 8 is a block diagram of the measuring system in Embodiment 2.

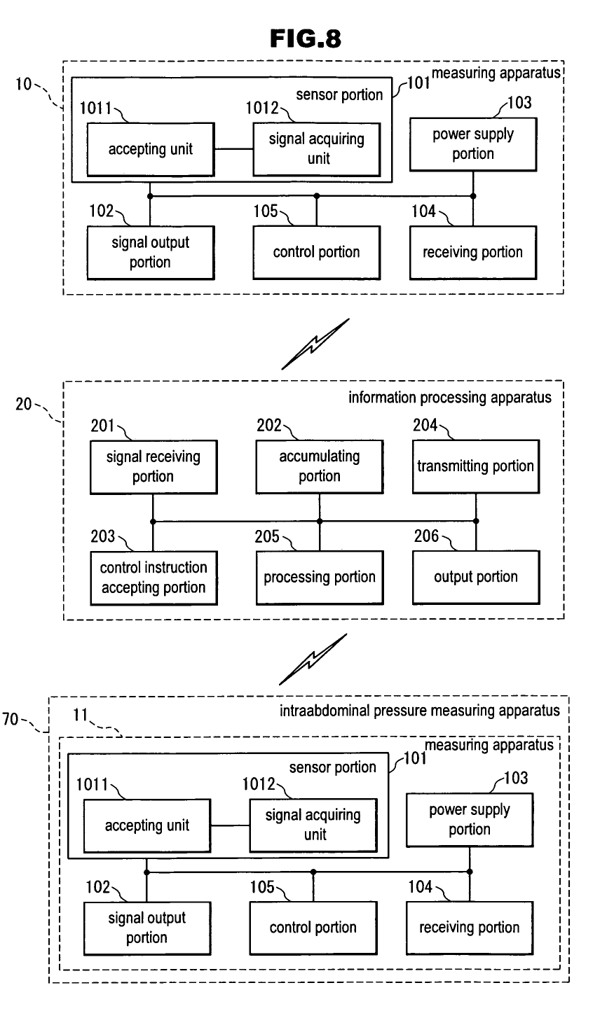

FIG. 9 is a view showing an arrangement example of the intraabdominal pressure measuring apparatus inside the living body.

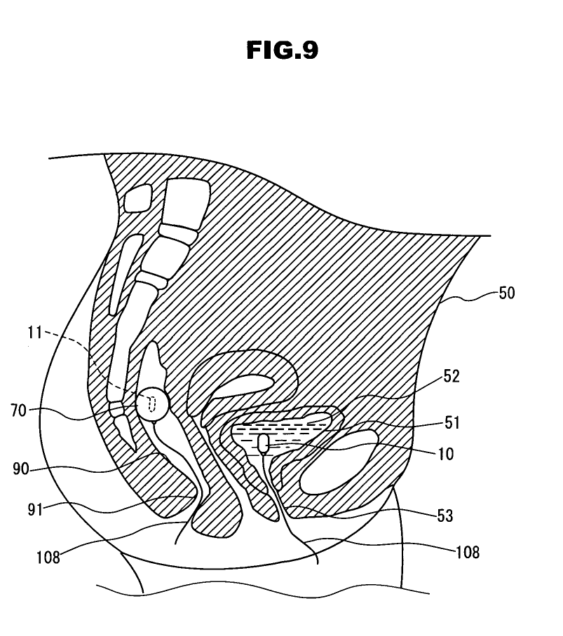

FIG. 10 is a graph showing a display example in the measuring system.

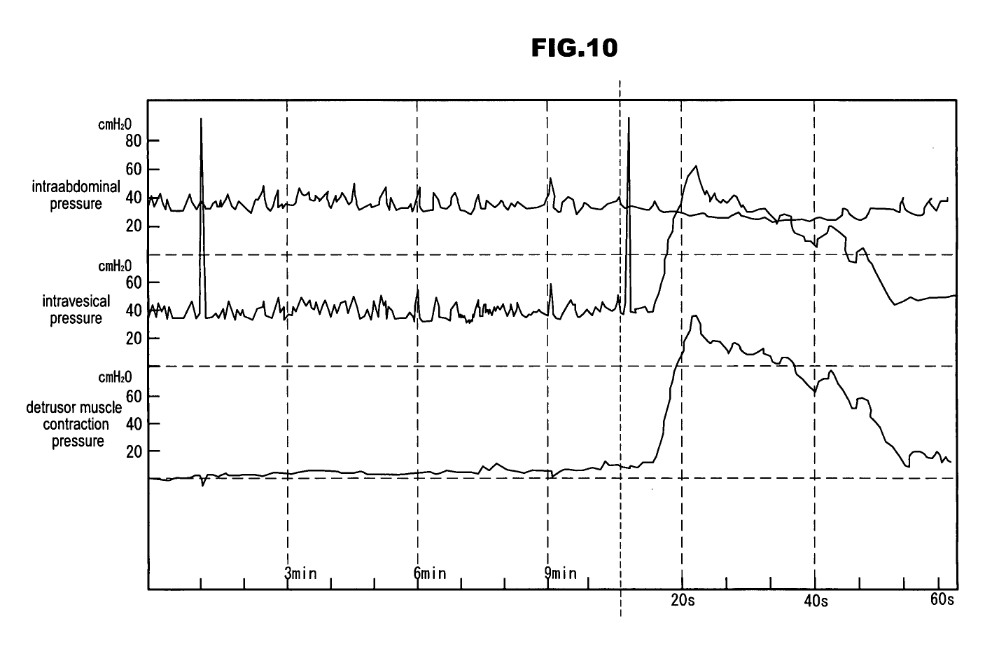

FIG. 11 is a view showing a modified example of the measuring apparatus.

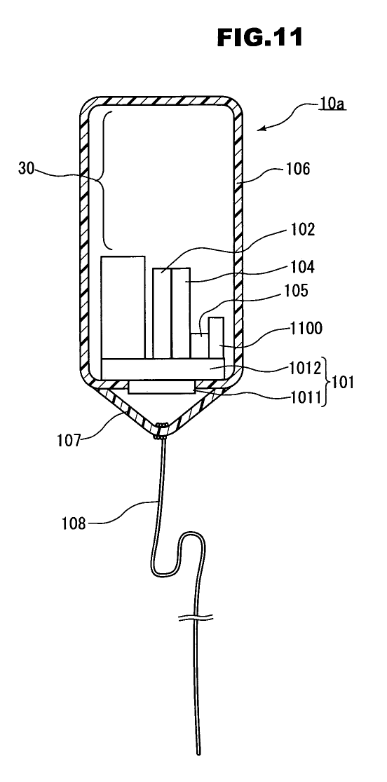

1. A measuring apparatus disposed inside the living body, comprising:
a sensor portion that has an accepting unit accepting a physical quantity that is to be measured and a signal acquiring unit acquiring a detection signal, which is a signal corresponding to the physical quantity accepted by the accepting unit;
a signal output portion that wirelessly outputs the detection signal to the exterior; and
a vessel in which at least the signal acquiring unit of the sensor portion and the signal output portion are sealed.

2. A measuring apparatus disposed inside the living body, comprising:
a sensor portion that has an accepting unit accepting a physical quantity that is to be measured and a signal acquiring unit acquiring a detection signal, which is a signal corresponding to the physical quantity accepted by the accepting unit;
an accumulating portion that accumulates the detection signal; and
a vessel in which at least the signal acquiring unit of the sensor portion and the accumulating portion are sealed.

3. The measuring apparatus according to claim 1, further comprising a power supply portion that supplies power to the signal acquiring unit of the sensor portion and the signal output portion.

4. The measuring apparatus according to claim 1, wherein a linear member, which is a member in the shape of a line that extends to the exterior, is attached to the vessel.

5. The measuring apparatus according to claim 1, wherein the accepting unit is exposed on the exterior of the vessel.

6. The measuring apparatus according to claim 5, further comprising a protector that is shaped so as to cover the accepting unit.

7. The measuring apparatus according to claim 1, wherein the measuring apparatus is disposed in a fluid inside the living body, and the specific gravity of the measuring apparatus is not greater than that of the fluid.

8. The measuring apparatus according to claim 1, wherein the measuring apparatus is disposed in a fluid inside the living body, and the measuring apparatus floats in the fluid.

9. The measuring apparatus according to claim 7,
wherein the accepting unit is disposed at a position displaced from the center of the vessel, and
the center of gravity of the measuring apparatus is positioned closer to the accepting unit.

10. The measuring apparatus according to claim 7,
wherein the accepting unit is disposed in a lower portion in the vessel, and
a space is provided in an upper portion in the vessel.

11. The measuring apparatus according to claim 7, wherein the accepting unit is disposed in the lower portion in the vessel.

12. The measuring apparatus according to claim 1, wherein the accepting unit is for accepting pressure.

13. The measuring apparatus according to claim 1, further comprising:
a receiving portion that receives a control signal, which is a signal wirelessly transmitted from the exterior and used for controlling the measuring apparatus; and
a control portion that controls the measuring apparatus based on the control signal.

14. The measuring apparatus according to claim 1, further comprising:
a flexible protection vessel; and
a fluid or gel that is sealed together with the vessel in the protection vessel.

15. The measuring apparatus according to claim 14, wherein a linear member that extends to the exterior is attached to the protection vessel.

16. A measuring system provided with a measuring apparatus disposed inside the living body and an information processing apparatus,
wherein the measuring apparatus comprises:
a sensor portion that has an accepting unit accepting a physical quantity that is to be measured and a signal acquiring unit acquiring a detection signal, which is a signal corresponding to the physical quantity accepted by the accepting unit;
a signal output portion that wirelessly outputs the detection signal to the exterior; and
a vessel in which at least the signal acquiring unit of the sensor portion and the signal output portion are sealed, and
the information processing apparatus comprises:
a signal receiving portion that receives the detection signal; and
an accumulating portion that accumulates the received detection signal.

17. The measuring system according to claim 16, wherein the information processing apparatus further comprises a processing portion that performs a given process on the detection signal accumulated by the accumulating portion.

18. The measuring system according to claim 16, wherein the information processing apparatus further comprises an output portion that outputs the detection signal accumulated by the accumulating portion or the detection signal processed by the processing portion.

19. The measuring system according to claim 16,
wherein the information processing apparatus further comprises:
a control instruction accepting portion that accepts a control instruction, which is an instruction for controlling the measuring apparatus; and
a transmitting portion that wirelessly transmits a control signal, which is a signal for controlling the measuring apparatus based on the control instruction, and
the measuring apparatus further comprises, in the vessel:
a receiving portion that receives the control signal transmitted from the information processing apparatus; and
a control portion that controls the measuring apparatus based on the control signal.
